(19) United States
(12) Patent Application Publication
(10) Pub. No.: US 2026/0047834 A1
(43) Pub. Date: Feb. 19, 2026

(54) INTERNAL WOUND TREATMENT SYSTEMS, METHODS, AND DEVICES

(71) Applicant: DEFENSE MEDICAL TECHNOLOGIES, INC., Tampa, FL (US)

(72) Inventors: Frederick J. THABET, Boca Raton, FL (US); Ravi KANAGALA, Boca Raton, FL (US); Mehdi El Messoussi GARTI, Boca Raton, FL (US)

(73) Assignee: DEFENSE MEDICAL TECHNOLOGIES, INC., Tampa, FL (US)

(21) Appl. No.: 19/102,238

(22) PCT Filed: Sep. 25, 2023
(86) PCT No.: PCT/US2023/075040

§ 371 Date: Feb. 7, 2025

Related U.S. Application Data
(60) Provisional application No. 63/409,488, filed on Sep. 23, 2022.
(60) Provisional application No. 63/489,095, filed on Mar. 8, 2023.

(51) Int. Cl. A61B 17/00; A61B 90/00

(52) U.S. Cl. CPC A61B 17/0057; A61B 90/06; A61B 2017/00026; A61B 2017/00084; A61B 2017/00115; A61B 2017/00557; A61B 2017/00893; A61B 2090/064

(57) ABSTRACT

Systems, methods, and devices for internal wound treatment include a hemostasis device. The hemostasis device has an insertion tube extending from a base portion. One or more openings are formed into a surface of the insertion tube. Additionally, the hemostasis device includes an inflation pump couplable to a proximal of the insertion tube for pumping air into an inner channel of the insertion tube. A wound channel balloon is disposed at least partially around the insertion tube such that an actuation of the inflation pump causes the air to flow from the inner channel through the one or more openings and inflate the wound channel balloon. The wound channel balloon can include a grooved elastic fiber, one or more bulges, and/or one or more branches. Furthermore, a collagen gel, an antibiotic, a blood clotting agent, an analgesic, stem cells, a sealant, and/or a lubricant can be disposed on the wound channel balloon.

### CROSS-REFERENCE TO RELATED APPLICATIONS

This application claims priority to U.S. Provisional Patent Application Ser. No. 63/409,488 filed Sep. 23, 2022 and titled “INTERNAL WOUND TREATMENT SYSTEMS, METHODS, AND DEVICES;” and U.S. Provisional Patent Application Ser. No. 63/489,095 filed Mar. 8, 2023 and titled “INTERNAL WOUND TREATMENT SYSTEMS, METHODS, AND DEVICES,” the entireties of which are incorporated herein by reference.

### BACKGROUND

Some wound treatment techniques performed in the field (e.g., away from a clinic setting) only provide topical treatment or treat only a portion of a wound. These types of treatments do not treat an interior portion of the wound and can be ineffective for treating deeper wounds (e.g., from ballistic projectiles). When the injury occurs in the field, more substantive treatment, such as internal stitches, are often delayed until the injured person is relocated to the clinic setting. Many injured people lose dangerous amounts of blood immediately following their injury and/or while awaiting transport to the clinic setting.

It is with these observations in mind, among others, that various aspects of the presently disclosed technology were conceived and developed

### SUMMARY

Systems, methods, and devices can address the aforementioned problems. For examples, a hemostasis device can include an insertion tube extending with a distal end and one or more openings formed into a surface of the insertion tube; an inflation pump couplable to the distal end of the insertion tube for pumping air into an inner channel of the insertion tube; and/or a wound channel balloon disposed at least partially around the insertion tube such that an actuation of the inflation pump causes the air to flow from the inner channel through the one or more openings and inflate the wound channel balloon.

In some instances, the device further includes a holding strap coupling to a first side of the insertion tube and a second side of the insertion tube. The insertion tube can have at least one of a flexible portion or an articulated portion. Also, the wound channel balloon can include expansion fingers configured to expand into wound features in response to the inflation pump inflating the wound channel balloon. The device can further include a gauge fluidly coupled to the insertion tube to indicate a pressure corresponding to an interior of the wound channel balloon. Furthermore, the device can include one or sensors to generate data related to one or more health parameters; and/or a wireless interface for transmitting the data related to one or more health parameters. For instance, the wireless interface can be a Bluetooth interface. Moreover, the device can include a global positioning system (GPS) system; and/or a cover such that removal of the cover from the insertion tube 108 triggers the GPS system to generate and transmit location data. The device can also include a collagen gel carrier agent disposed on an outer surface of the wound channel balloon, the carrier agent including at least one of: an antibiotic, an analgesic, a coagulant, or a glue. The carrier agent can be a bovine-based collagen matrix, and/or the carrier agent can include a mollusk-based glue.

In some examples, a hemostasis device includes a flexible insertion tube including one or more air pathway openings fluidly coupling an inner channel of the flexible insertion tube to an inflation space defined between an outer surface of the flexible insertion tube and a wound channel balloon; the wound channel balloon disposed at least partially over the flexible insertion tube; a first coupling mechanism for fluidly coupling an inflation pump to the flexible insertion tube; and/or a second coupling mechanism for removably coupling a cover over the flexible insertion tube. The device can also include the inflation pump couplable to a first end of the flexible insertion tube such that the inflation pump is fluidly coupled to the inflation space between the outer surface of the flexible insertion tube and the wound channel balloon, actuations of the inflation pump expanding the inflation space. In some scenarios, the device includes one or more gripping arms extending from the hemostasis device between the first coupling mechanism and the second coupling mechanism providing one-handed removal of the hemostasis device. Additionally, the cover can include one or more gripping arms providing one-handed access for pulling the cover away from the wound channel balloon and the flexible insertion tube.

In some instances, a method of performing wound hemostasis includes inserting a distal end of an insertion tube into a wound channel of a patient, the distal end at least partially covered by a wound channel balloon; inflating the wound channel balloon by pumping air into the insertion tube such that the wound channel balloon is an at least partially inflated wound channel balloon; and/or occluding a blood flow from an interior of the wound channel by applying a pressure against the interior of the wound channel with the at least partially inflated wound channel balloon.

In some examples, inflating the wound channel balloon can include actuating an inflation pump fluidly coupled to the wound channel balloon; and/or the method can further include detaching the inflation pump from the insertion tube. An outer surface of the wound channel balloon can be coated with a collagen-based matrix and occluding the blood flow can further include providing an antibiotic and an analgesic to the interior of the wound channel with the collagen-based matrix. Moreover, occluding the blood flow of the interior of the wound channel can further include at least one of coagulating the blood flow with a coagulant carried by the collagen-based matrix; and/or sealing the interior of the wound channel with a mollusk-based glue. Furthermore, occluding the blood flow of the interior of the wound channel can include securing a holding strap around a portion of a person receiving a device including the insertion tube, the holding strap being coupled to a first side and a second side of the device.

### BRIEF DESCRIPTION OF THE DRAWINGS

The foregoing summary, as well as the following detailed description, will be better understood when read in conjunction with the appended drawings. For the purpose of illustration, there is shown in the drawings certain embodiments of the disclosed subject matter. It should be understood, however, that the disclosed subject matter is not limited to the precise embodiments and features shown. The accompanying drawings, which are incorporated in and constitute a part of this specification, illustrate an implementation of systems and methods consistent with the disclosed subject matter and, together with the description, serves to explain advantages and principles consistent with the disclosed subject matter, in which:

FIG. 1 illustrates an example system for performing a hemostasis procedure with a hemostasis device by depicting multiple top views of an injured leg section, which can form at least a portion of the system(s) disclosed herein;

FIG. 2 illustrates an example system for performing a hemostasis procedure with a hemostasis device by depicting multiple top views of an injured leg section, which can be combined with or form a portion of the system(s) disclosed herein;

FIG. 3 illustrates an example hemostasis device in an uninflated state, which can form at least a portion of the system(s) disclosed herein;

FIG. 4 illustrates an example hemostasis device in an inflated state, which can form at least a portion of the system(s) disclosed herein;

FIG. 5 illustrates an example hemostasis device transitionable between an inflated state and a deflated state and with a cover in the deflated state, which can form at least a portion of the system(s) disclosed herein;

FIG. 6 illustrates an example hemostasis device with a holding strap, which can form at least a portion of the system(s) disclosed herein;

FIG. 7 illustrates an example hemostasis device with a body having one or more gripping portions, one or more coupling mechanisms, and/or a cover, which can form at least a portion of the system(s) disclosed herein;

FIG. 8 illustrates an example hemostasis device with a body portion coupled to a holding strap, which can form at least a portion of the system(s) disclosed herein;

FIG. 9 illustrates an example hemostasis device with a body portion coupled to a holding strap, which can form at least a portion of the system(s) depicted in FIGS. 1 and 2;

FIG. 10 illustrates an example method for using a hemostasis device to occlude a blood flow, which can be performed by the system(s) depicted in FIGS. 1 and 2 and/or the devices depicted in FIGS. 3-9 and 11A-13;

FIGS. 11A-11C illustrate an example hemostasis device and components thereof, which can form at least a portion of the system(s) disclosed herein;

FIGS. 12A and 12B illustrate an example system for performing a hemostasis procedure with a hemostasis device for an injured leg, including multiple procedure steps, which can form a part of the method of FIG. 10 and/or be performed by the systems disclosed herein;

FIG. 13 illustrates an example system for performing a hemostasis procedure at an injured abdomen, which can form at last a portion of the system(s) disclosed herein;

FIG. 14 illustrates an example hemostasis device with a grooved outer layer to hold a carrier agent, which can form at last a portion of the system(s) disclosed herein; and

FIGS. 15A-15D illustrate an example hemostasis device and components thereof, which can form at least a portion of the system(s) disclosed herein

### DETAILED DESCRIPTION

It will be appreciated that for simplicity and clarity of illustration, where appropriate, reference numerals have been repeated among the different figures to indicate corresponding or analogous elements. In addition, numerous specific details are set forth in order to provide a thorough understanding of the embodiments described herein. However, it will be understood by those of ordinary skill in the art that the embodiments described herein can be practiced without these specific details. In other instances, methods, procedures and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present technological concepts.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the presently disclosed technology or the appended claims. Further, it should be understood that any one of the features of the presently disclosed technology may be used separately or in combination with other features. Other systems, methods, features, and advantages of the presently disclosed technology will be, or become, apparent to one with skill in the art upon examination of the figures and the detailed description. It is intended that all such additional systems, methods, features, and advantages be included within this description, be within the scope of the presently disclosed technology, and be protected by the accompanying claims.

Further, as the presently disclosed technology is susceptible to embodiments of many different forms, it is intended that the present concept be considered as an example of the principles of the presently disclosed technology and not intended to limit the presently disclosed technology to the specific embodiments shown and described. Any one of the features of the presently disclosed technology may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments, but is not necessarily included. Thus, the presently disclosed technology may include a variety of combinations and/or integrations of the embodiments described herein. Additionally, all aspects of the present concept, as described herein, are not essential for its practice. Likewise, other systems, methods, features, and advantages of the presently disclosed technology will be, or become, apparent to one with skill in the art upon examination of the figures and the description. It is intended that all such additional systems, methods, features, and advantages be included within this description, be within the scope of the presently disclosed technology, and be encompassed by the claims.

Any term of degree such as, but not limited to, “substantially,” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50°includes all values from 16.6°to 150°.

The term “coupled” is defined as connected, whether directly or indirectly through intervening components, and is not necessarily limited to physical connections. The connection can be such that the objects are permanently connected or releasably connected. The terms “comprising,” “including” and “having” are used interchangeably in this disclosure. The terms “comprising,” “including” and “having” mean to include, but not necessarily be limited to the things so described. The term “real-time”or “real time”means substantially instantaneously.

Lastly, the terms “or” and “and/or,” as used herein, are to be interpreted as inclusive or meaning any one or any combination. Therefore, “A, B, or C” or “A, B, and/or C” mean any of the following: “A,” “B,” or “C”; “A and B”; “A and C”; “B and C”; “A, B and C.” An exception to this definition will occur only when a combination of elements, functions, steps or acts are in some way inherently mutually exclusive.

The systems, methods, and devices disclosed herein include a hemostasis device, for instance, to be used in emergency scenarios. The hemostasis device can include a portable, self-contained, handheld, easy to deploy, single use, and/or self-administered wound channel balloon coupled to an insertion tube. Upon experiencing an injury that forms a wound channel (e.g., bullet, shrapnel, etc.), the hemostasis device can be removed from packaging (e.g., a cover pulled off with one hand), and the insertion tube can be inserted into the wound channel. Direct pressure can be delivered internally to the inner walls of the wound channel (e.g., inside the injured portion of the patient's body) by inflating the wound channel balloon with an inflation pump. The wound channel balloon can be formed of a very pliable, forgiving material that can minimize damage to the internal tissue of the body as it is inflated to match the trajectory of the bullet or object that caused the injury. The wound channel balloon can be inflated to a pressure that is optimal to occlude the flow of blood from damaged arteries and veins without being so aggressive that it causes further damage to the surrounding tissue. Furthermore, a carrier agent (e.g., a collagen matrix) on an exterior of the wound channel balloon can provide medicine (e.g., antibiotics, analgesics, coagulants), and/or additional structural support (e.g., glue) for treating the wound. As such, the blood flow can be occluded and the injured person's life prolonged (e.g., by preventing critical levels of blood loss) until they can receive more complete treatment (e.g., clinical treatment) for the wound.

Additionally, in some instances, the hemostasis device can include a controller to receive sensor data from the internal wound site and a wireless transmitter to send the sensor data to a remote device. Accordingly, health parameters (e.g., blood pressure, heart rate, etc.) of the person being treated can be monitored. Location data can be generated and transmitted from the hemostasis device, using a secure communication line, for remote tracking of the injured person using the hemostasis device (responsive to the hemostasis device being activated).

Additional advantages of the systems, methods, and devices discussed herein will become apparent from the detailed description below.

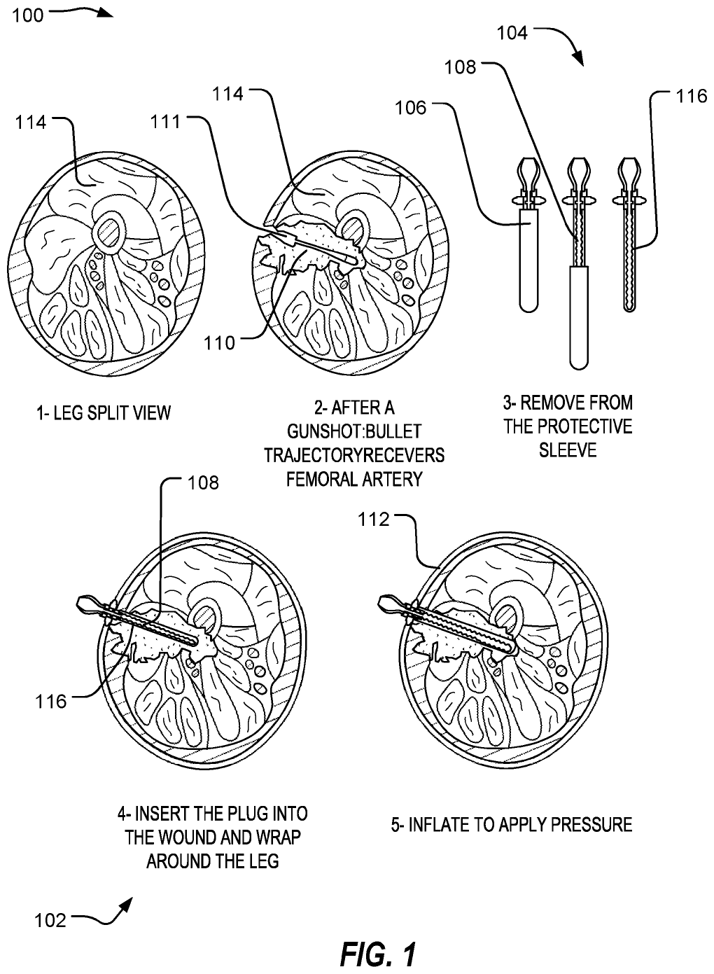

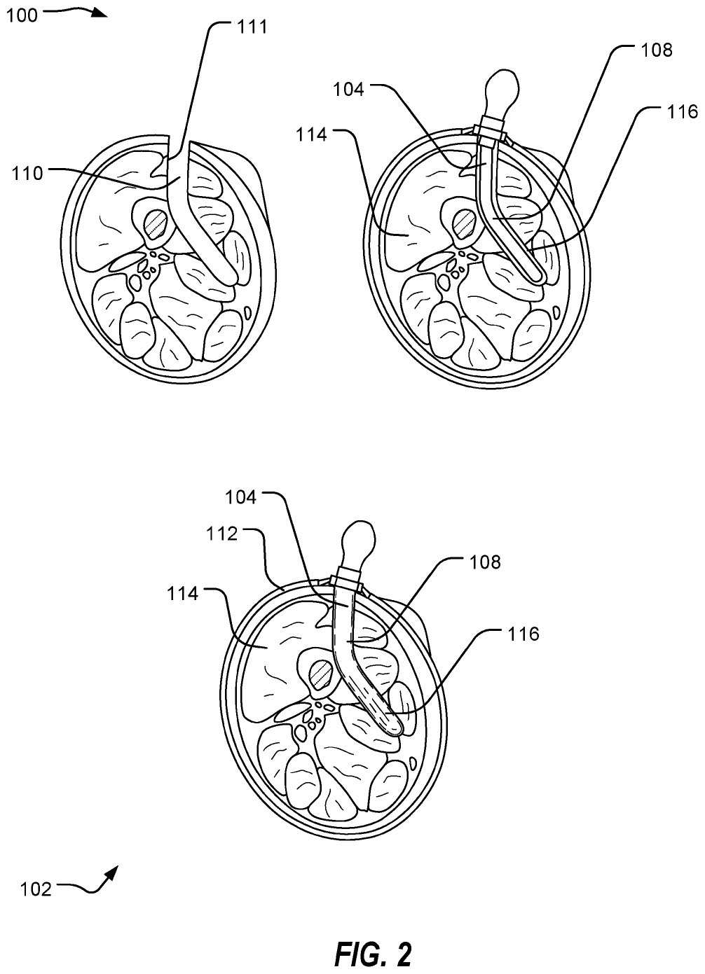

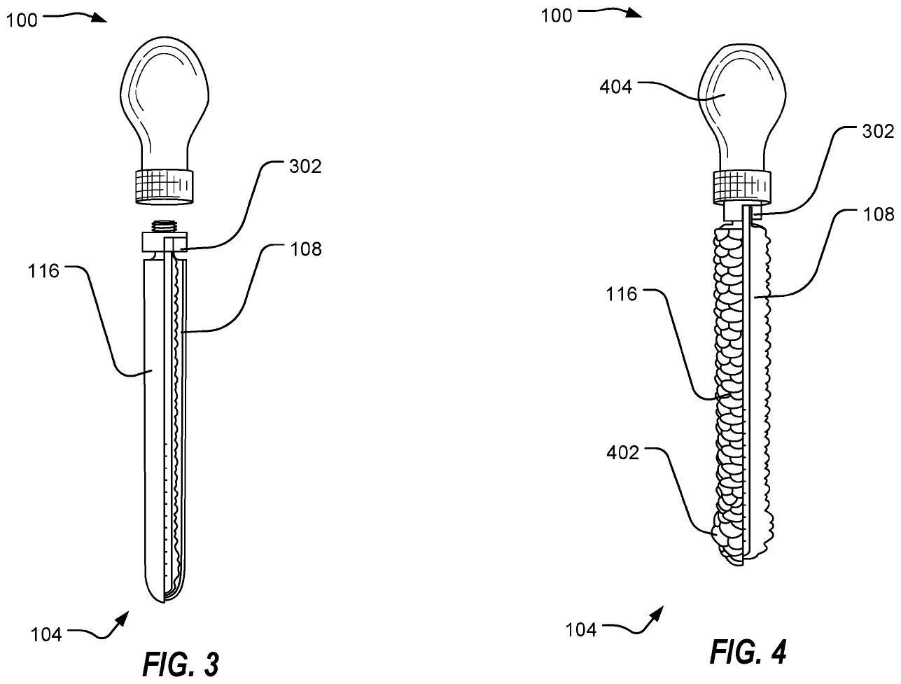

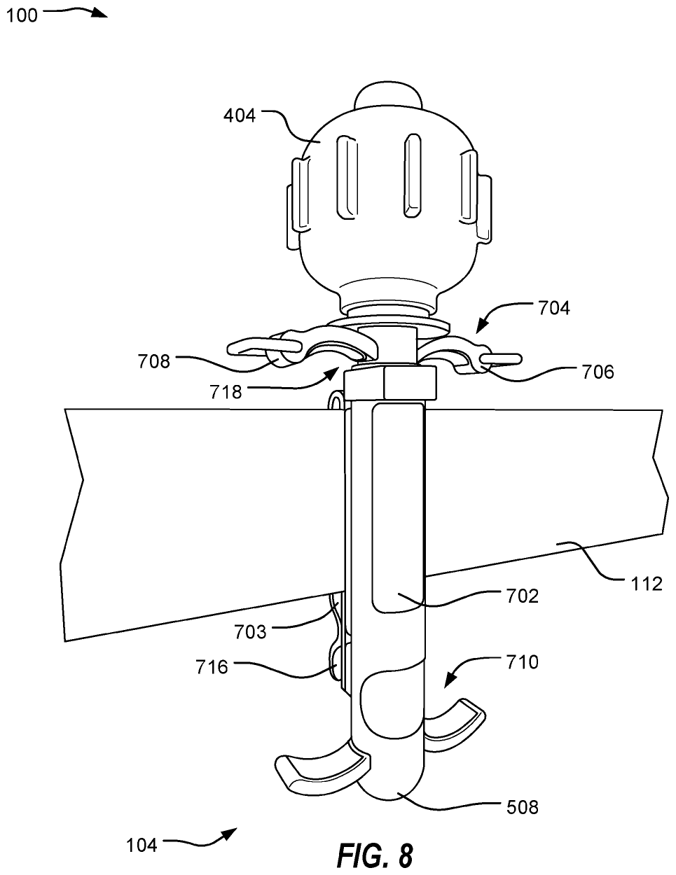

FIGS. 1-15D illustrate various embodiments of example internal hemostasis device(s), system(s), and/or method(s) for occluding blood flow from a wound (e.g., in an emergency environment). FIGS. 1 and 2 depict various operations of a method for using the internal hemostasis device and FIGS. 3-9 illustrate various components of the emergency hemostasis device.

Turning to FIGS. 1 and 2, top views of a leg section including a wound are depicted. A system 100 can include a method 102 of using a hemostasis device 104, which can include removing a protective sleeve 106 from the hemostasis device 104 (e.g., an insertion tube 108 of the hemostasis device 104) from a protective sleeve 106. The method 102 can include inserting a plug or insertion tube 108 of the hemostasis device 104 into a wound 110 and wrapping a strap 112 or other plug holder around a portion of the patient's body 114 (e.g., the wounded leg), securing the insertion tube 108 place. The method 102 can include inflating a wound channel balloon 116 to cause pressure to be applied into the wound 110, thus occluding blood flow from the wound 110, as discussed in greater detail below, various portions of the wound channel balloon 116 (e.g., fingers, protrusions, bulges, etc.) can expand into the crevices of damaged vein arteries, capillaries, muscle tissue, and/or bone losing blood from the wound 110, applying pressure which stops the blood flow.

Turning to FIGS. 3-9, various components of the emergency or handheld internal hemostasis device 104 are depicted. Any of the features discussed regarding FIGS. 3-9 can be combined together and/or combined with those discussed herein regarding FIGS. 1, 2 and/or 10-15D.

In some examples, the hemostasis device 104 includes a wound channel balloon 116 such that direct pressure can be delivered internally to the walls of the wound channel 111 by using an inflatable balloon (e.g., the wound channel balloon 116). The wound channel balloon 116 can be at least partially disposed around an insertion tube 108 with an end coupling to top portion 302 of the insertion tube 108 (e.g., at a body portion of the hemostasis device 104). The wound channel balloon 116 can be made of a very pliable, forgiving material that minimizes damage to the internal tissue of the body as it is inflated to match the trajectory of the bullet or object that causes the injury. The wound channel balloon 116 can be inflated to a pressure that is optimal to occlude the flow of blood from damaged arteries and veins without being so aggressive that it will cause further damage to the surrounding tissue.

In some instances, the hemostasis device 104 includes an attached pressure gauge which can be a simple pressure gauge attached to the proximal components of device so an operator can see an inflation pressure measurement of the balloon. The pressure gauge can be color coated with green, yellow, and red indications to let the operator visualize easily if they are inflating the balloon to an unsafe level and/or an optimal pressure.

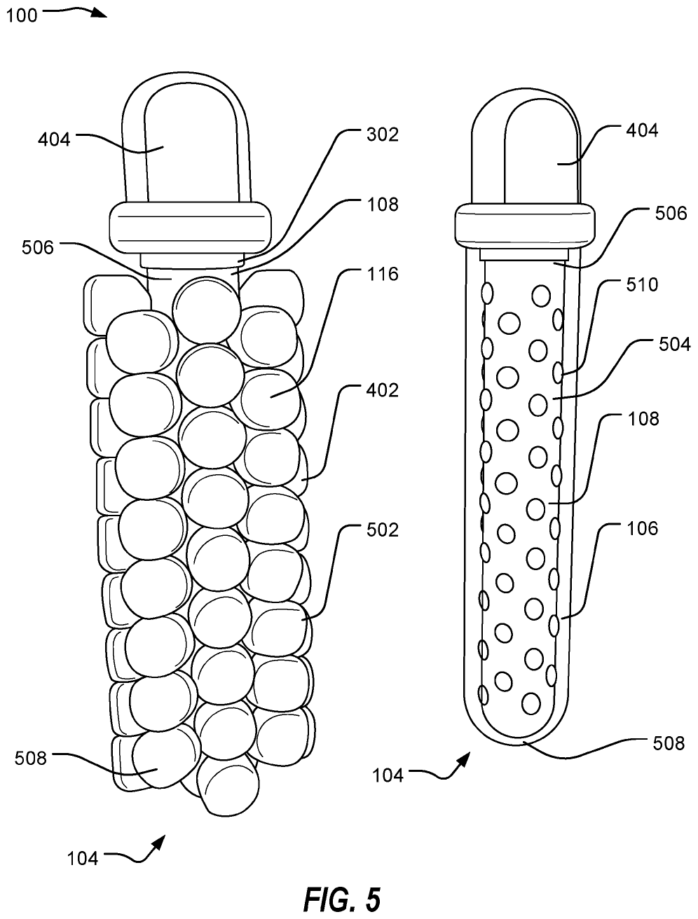

In some examples, the hemostasis device 104 includes balloon fingers 402 (e.g., as depicted in FIGS. 4 and 5). The wound channel balloon 116 can also feature finger-like protrusions 502 extending at least partially perpendicularly from a main body 504 of the wound channel balloon 116 which inflate and follow the path of small tributaries that come off the main wound channel 111. The idea behind the inflatable finger-like protrusions 502 is once they find a small tributary, they can inflate to occlude the bleeding along those smaller channels.

In some instances, the hemostasis device 104 includes an insertion tube 108. The wound channel balloon 116 can be attached around an insertion tube 108 with a (e.g., red) squeeze/inflation bulb or pump 404 at the proximal end of the insertion tube 108. The insertion tube 108 can provide a way for the operator to expand the wound channel balloon 116 via the red squeeze bulb 404 located at the proximal end 506 of the insertion tube 108 by squeezing his or her fingers together.

In some examples, the hemostasis device 104 includes an articulating and/or flexible insertion tube 108. The insertion tube 108 can be flexible and/or a distal end 508 or third of the tube can be able to articulate. The flexible insertion tube 108 along with the articulating tip can allow the operator to migrate the flexible insertion tube 108 along a twisting wound channel 111 so that the insertion tube 108, with the attached wound channel balloon 116, can be seated along the same path the bullet or penetrating object took.

In some scenarios, the hemostasis device 104 includes a fenestrated and/or perforated insertion tube 108. The insertion tube 108 can be fenestrated or perforated with one or more openings 510 on a surface of the insertion tube 108 to allow the air to inflate the wound channel balloon 116 as a bulb 404 is depressed. For instance, an inflation space between an outer surface of the insertion tube 108 and an inner surface of the wound channel balloon 116 can be fluidly coupled to an inner channel of the insertion tube 108 via the one or more openings 510. As the air enters the wound channel balloon 116 through the perforations/openings 510 in the insertion tube 108, the soft, pliable wound channel balloon 116 can (e.g., with finger-like protrusions 502) expand into the wound channels 111 created by the bullet or penetrating object.

In some instances, the hemostasis device 104 includes an inflation bulb or pump 404 (e.g., a bright red inflation bulb). The inflation pump 404 can be placed on top of the proximal end 506 of the insertion tube 108. The inflation pump 404 can be designed to inflate the wound channel balloon 116 attached to the outside of the insertion tube 108 via compressions (e.g., hand pumps) provided by an operator. The inflation pump 404 can deliver enough air under force to inflate the wound channel balloon 116 effectively with wound channel balloon 116 deployed into the wound 110 of the patient. The inflation pump 404 can be coated with a high-friction material or sticky material (e.g., Shark Skin, Lizard Skin, etc.) that creates a surface the operator can easily grip and find traction through blood, perspiration, water, mud, or any other compromising substance that finds its way onto the operator's hands or onto the hemostasis device 104 itself.

In some examples, the hemostasis device 104 includes a one-way valve, for instance, inset into the proximal opening of the mouth of the insertion tube 108 (e.g., at a first connector for coupling a body portion of the hemostasis device 104 to the inflation pump 404). Additionally or alternatively, the one-way valve can be seated directly distal to the end of the inflation pump 404 that attaches to the proximal end 506 of the insertion tube 108. As the inflation pump 404 is depressed, air can be forced into the wound channel balloon 116 causing it to inflate. Air can be prevented from escaping the wound channel balloon 116 due to the one-way valve. When the injured person is able to get to a physician that can surgically intervene to save their lives, the surgeon can twist the red inflation pump 404 off the proximal end of the insertion tube 108 and press down on the one-way valve to release the air and deflate the wound channel balloon 116. The hemostasis device 104 can then be safely removed from the wound channel 111 of the injured person.

In some instances, the hemostasis device 104 includes a meshed insertion material (e.g., or sock). The meshed insertion sock can be attached onto an outer surface of the wound channel balloon 116. The meshed insertion sock can be made of an elastic, absorbent, fibrous, and/or gauze like material that can expand into the crevices of the wound channel 111 as the wound channel balloon 116 inflates and expands. The meshed insertion sock can be heavily coated in a wound-healing and/or health promoting gel or material called Cryogen. One purpose of the meshed sock can be to carry the Cryogen into the wound channel 111 so that the Cryogel coats the entire surface of the wound channel 111 in the Cryogel material. The Cryogel can be a collagen based gelatinous matrix. Cryogel can act as a carrier for antibiotics, morphine, coagulants, and/or a mollusk glue to cause adhesion to the walls of the wound channel 111 as well as the open ends of blood vessels. Cryogel can be a fortified bonding and/or coating agent that is designed to seal, coat, and carry (e.g., antibiotics, analgesics, and/or hemostatic agents). In some instances, the wound channel balloon 116 and/or a material layered over the wound channel material (e.g., with the Cryogel) can include a structural shaping component (e.g., a seam, flap, a bulge, a contour, a support rod, or other protrusion-like structure etc.) forming one or more expansions that act as fingers to extend away from the main channel of the insertion tube 108 (e.g., finger expansions). In some examples, (e.g., as shown in FIG. 5) a portion of the meshed insertion material and/or the wound channel balloon 116 can be initially disposed inside the insertion tube 108. Upon being inflated, the meshed insertion material and/or the wound channel balloon 116 can expand through one or more openings 510 in a side of the insertion tube 108 (e.g., a plurality or array of perforations), creating the expansion finger-like protrusions 502. The Cryogel can be mixed with and/or coated onto the meshed material. Additionally or alternatively, the Cryogel can be at least partially or fully coated directly onto the outer surface of the insertion tube 108.

In some instances, the Cryogel can seal the ends of lacerated, punctured and torn blood vessels to help prevent catastrophic blood loss. The Cryogel can be designed to be smeared over an entire wound's surface area so that the full surface of the wound 110 is covered and coated (e.g., or a portion of the wound surface). The Cryogel can be designed to be a carrier of other medications directly to the wound surface. By way of example, the Cryogel can carry antibiotics such as vancomycin, gentamicin, and cefazolin (Ancef). The Cryogel can be designed to carry painkilling medications directly to the wound surface such as morphine to help deaden the pain of the trauma. Furthermore, the Cryogel can be designed to carry hemostatic agents directly to the wound surface such as Avitene, Thrombin and Fibrin Sealants. These agents can be designed to cause hemostasis with open vessels. Moreover, in some instances the Cryogel can operate as a foam and/or can form a carrier matrix on an outside surface of the balloon and/or the insertion tube 108. In some instances, the Cryogel includes a bovine collagen. The Cryogel can include a mix of antibiotic, coagulant, analgesic, and glue (e.g., a synthetic, animal, and/or mollusk-based glue).

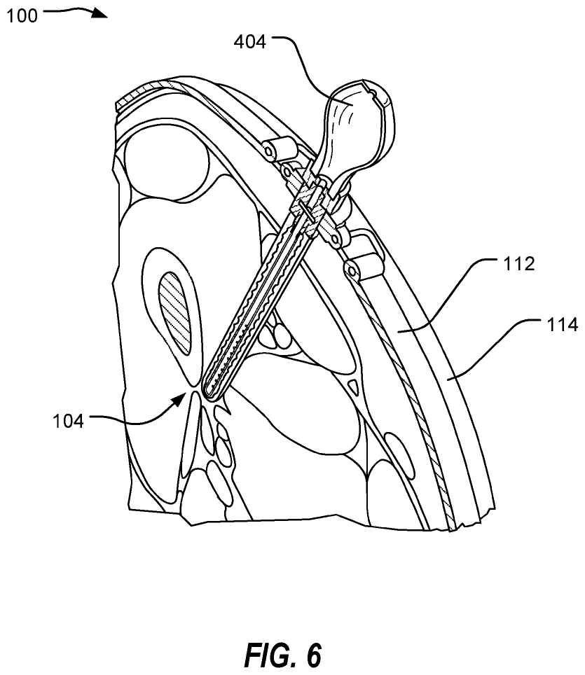

In some examples, the hemostasis device 104 can include a holding strap 112, such as a tourniquet holding strap (e.g., using hook-and-loop attachment mechanisms), as depicted in FIG. 6. The tourniquet and/or holding strap 112 can be included in the device package and can be designed to wrap around a limb or the body part where the hemostasis device 104 has been applied. The holding strap 112 can maintain the hemostasis device 104 in place. For instance, the holding strap 112 can help maintain the hemostasis device 104 in its inflated position inside the wound channel 111. The holding strap 112 can be designed to maintain a constant and stabile position of the hemostasis device 104 so that movement does not dislodge the device. The holding strap 112 can also help maintain a constant pressure on the wound 110: The holding strap 112 can also be designed to help maintain constant pressure on the damaged areas of the body to help with hemostasis. The holding strap 112 can be used as a Tourniquet. In other words, the hemostasis device 104 can have multiple redundant systems so that the wounded individual has the maximum opportunity to survive the wound 110 with every possible advantage. The holding strap 112 can be used with a stick as a tourniquet in scenarios where the limb has been severed and/or is severely bleeding.

In some examples, the hemostasis device 104 includes a portable, sterile, and/or self-contained, packaging container. The hemostasis device 104 can be contained in its own portable, sterile, self-contained package that can be easily carried on any individual. The hemostasis device 104 can be lightweight and small enough so that a soldier can carry as many as five hemostasis devices 104 on their person at any one time. The packaging can carry a two-year shelf life and be deployable by the injured person if necessary. For instance, the hemostasis device 104 can be inserted and packaged into a test tube style container that is designed to maintain sterility as well as house the Cryogel. In some scenarios, the hemostasis device 104 includes a carrying cap.

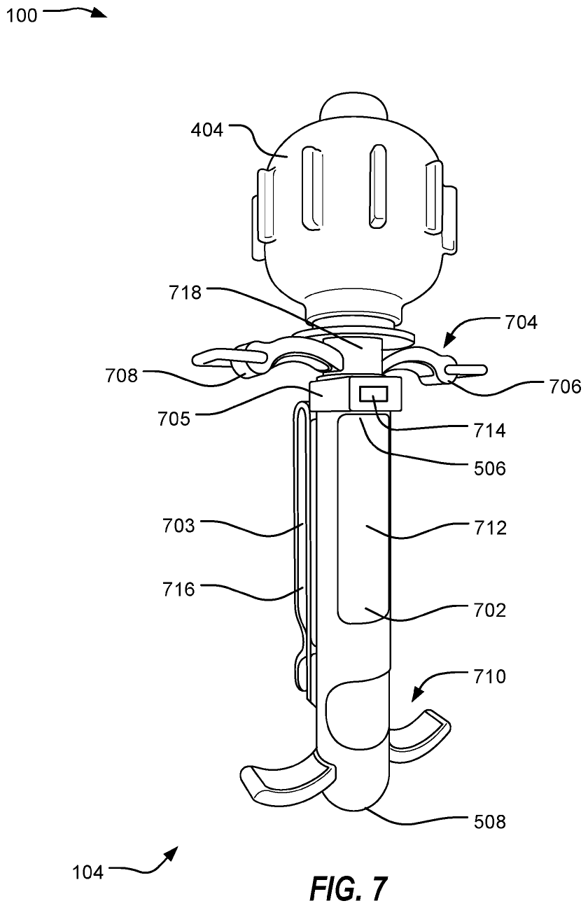

For instance, as shown in FIGS. 7-9, the wound channel balloon 116 and top of the hemostasis device 104 can be contained in a sterile protective carrying cap 702 (e.g., the protective sleeve 106) that can have a carrying clip 703 so that the hemostasis device 104 can be attached to the clothing of a soldier, stowed in a pocket, kept in a medic's satchel and so forth. The hemostasis device 104 can have a ring or pin attached to the top of the carrying cap 702 so that a wounded soldier can pull the carrying cap 702 off with his teeth if one hand is compromised.

In some instances, the hemostasis device 104 can include a computing device 703 such as a processor (e.g., a controller or microcontroller) and/or one or more memory devices storing non-transitory computer-readable instructions that, when executed by the one or more processors, cause the hemostasis device 104 to perform various steps. For instance, the computing components of the hemostasis device 104 can collect data from one or sensors built into the insertion tube 108 and/or the wound channel balloon 116. The sensors can receive and/or determine data detected at the wound site (e.g., the internal wound 110 inside the patient), such as impedance, blood pressure, body temperature, heart rate, glucose levels, blood type, or other health parameters. The computing device can include a communication port, such as a wireless interface (e.g., Bluetooth, Wi-Fi, Near-Field Communication, cellular, satellite, combinations thereof, and the like) to transmit the collected data to a remote receiver (e.g., at a base camp, vehicle, remote server, mobile device, etc.).

In some examples, one or more sensors can be used to make measurements and generate data associated with a health status patient. The electrical impedance and/or pressure of fluid, such as blood and/or oxygen, can be measured and used to assess a current health status and/or predict a future health status of a patient using the present concept. For instance, the electrical impedance or resistivity measurement of the blood can be used to determine a variety of real-time health statuses or conditions of the patient, such as whether the patient is bleeding (e.g., whether the patient has a wound 110), whether the patient is hemodynamically stable, and/or a severity of the bleeding. Furthermore, an impedance measurement can indicate a heart failure and/or fluid overload of the heart (e.g., based on a measurement of 60 ohms or greater), and/or an impedance measurement can indicate a low likelihood of fluid overload (e.g., a measurement of 30 ohms or less). In other words, a low impedance measurement can indicate fluid buildup and/or a worsening heart failure whereas a higher impedance can indicate that the patient is dry. One or more impedance measurement(s) can be used to assess other cardiac statuses. For a patient with a pacemaker, a high impedance measurement can indicate a lead fracture of the pacemaker, and a low impedance measurement can indicate an insulation break in the pacer lead. Additionally or alternatively, an impedance measurement can be used, according to the techniques discussed herein, to determine a presence of a skull bleed, a severity of skull bleed, and/or a stopping of a skull bleed.

In some scenarios, a higher severity of bleeding can correspond to a lower impedance measurement. For instance, a fracture or insulation brake may have an impedance range between 350 ohms and 1400 ohms, with the measurements near the 350 ohm end of the range corresponding to more severe bleeding and measurements near or greater than the 1400 ohm end of the range corresponding to less severe bleeding. By way of example, an impedance measurement and/or a change in measured impedance values can indicate that bleeding has stopped (e.g., a measured increase of impedance). As such a severity of bleeding and/or a degree of trauma can be determined in the field using the sensor measurements of the hemostasis device 104 (e.g., with changes detected and presented in real-time), rather than using other types of monitoring systems that may be more bulky or inaccessible in the field.

In some instances, an impedance value can be detected, measured, or otherwise determined by measuring an electrical difference at two or more measurement locations at the body of the patient. For instance, the hemostasis device 104 may be inserted at a first measurement location, for instance, at the wound 110 of a patient. A probe communicatively coupled to the hemostasis device 104 can be inserted into the patient at a second location (e.g., via a nasogastric (NG) tube or a needle), such that the electrical difference between the first measurement location and the second measurement location is measured with the hemostasis device 104 and the probe. In some scenarios, the first measurement location can be at the cranial cavity of the patient and a second measurement location can be at a nasal cavity of the patient using an NG tube. The NG tube receiving the probe for the second measurement location can be part of an NG apparatus for delivering medications and nutrition.

Understanding the health status of the patient (e.g., using the one or more impedance measurements) can be used to determine whether emergency action is required to prevent the patient from going into shock and/or dying. Furthermore, the present concept can utilize one or more predetermined thresholds to determine whether the one or more signals associated with the patient are within an acceptable range or outside of the acceptable range. For instance, particular impedance thresholds can correspond to different degrees of bleeding severity, whether the patient is hemodynamically stable, and/or whether the bleeding has stopped, as discussed above.

The hemostasis device 104 can also transmit location data (e.g., GPS data) collected by the computer 705 to a remote device so that the person having received the hemostasis device 104 is trackable to the remote device. In some instances, the health parameters may be determined and/or stored locally at the hemostasis device 104, and may be retrieved by a device in proximity to the hemostasis device 104. In these ways, the hemostasis device 104 can transmit useful health parameters, collected in real-time from the wound site, to device(s) associated with the medical personnel treating the patient (e.g., to assist in determining critical treatment steps) and/or to personnel locating and/or rendezvousing with the person who received the hemostasis device 104. Additionally or alternatively, the hemostasis device 104 can be used in a clinic setting to occlude blood flow (e.g., in an operating room and/or an emergency room). As such, the hemostasis device 104 can communicate the sensor data and/or the health parameters to one or more clinic devices using various wired or wireless connections. Furthermore, one or more displays (e.g., of the hemostasis device 104 and/or of a device remote from the hemostasis device 104) can present graphical indications (e.g., icons, alphanumeric symbols, graphs, images, sounds, etc.) of at least one of the impedance measurement value; a corresponding bleeding rate, a plurality of sequential impedance values; a corresponding change of a bleeding rate over time, whether the impedance value or the plurality of impedance values is above or below a predetermined threshold indicative of one or more health statuses or conditions (e.g., as discussed above); whether the impedance value or corresponding bleeding severity is increasing or decreasing; or whether the impedance value indicates the bleeding has stopped.

In some scenarios, the hemostasis device 104 can include one or more bifurcated grip arms 702, such as a first one or more grip arms 704 (e.g., a first side arm 706 and a second side arm 708) extending from the top portion of the insertion tube 108 near a connector for the wound channel balloon 116. This first grip arm(s) 704 can provide a gripping surface for pulling the hemostasis device 104 out of the wound 110 to perform subsequent medical treatment. The hemostasis device 104 can also include a second one or more gripping arms 710 at the distal end of a shell, cover, or housing 712 (e.g., the protective sleeve 106). The shell, cover, or housing 712 can be easily pulled off of the insertion tube 108 and the wound channel balloon 116 by grabbing the second grip arm(s) 710. These positionings of the first grip arm(s) 704 and/or the second grip arm(s) 710 can provide one-handed access for self-administration of the hemostasis device 104, for instance, in scenarios where the other hand or arm of person administering the hemostasis device 104 is injured or busy holding something.

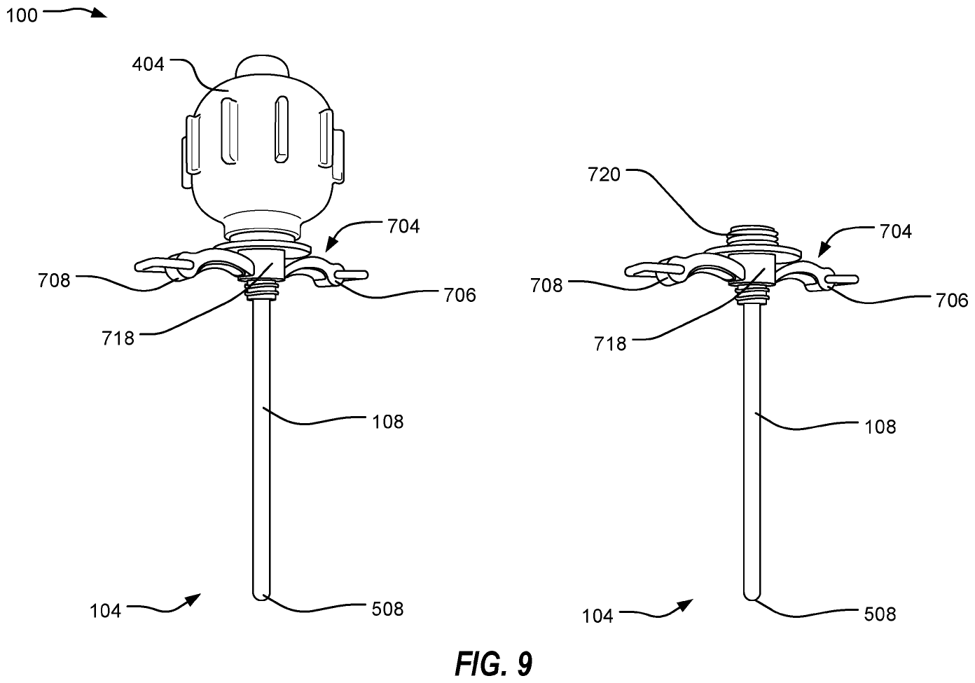

Furthermore, the hemostasis device 104 can include a GPS tracker 714 or other type of computing device (e.g., as discussed above) with one or more LEDs indicating if the hemostasis device 104 is active, ready for use, being used, ready to be pulled out, etc. A guide loop 716 can attach to the insertion tube 108 for guiding the holding strap 112 (e.g., attached to the insertion tube 108 at top portion of the insertion tube 108). In some examples, a jacket or coating on the inflation pump 404 can form a grip made of a “Lizard Skin” material. Additionally or alternatively, as shown in FIG. 9, the inflation pump 404 can be detachable from a body portion 718 of the insertion tube 108, for instance, to make the hemostasis device 104 flusher with the human body upon inserting it into the wound 110. In some examples, a cap can be placed over the top threaded opening 720 at the top of the body portion 718 of the hemostasis device 104 to cover the opening 720. The shell 712 over the insertion tube 108 can also be removed from a second coupling mechanism (e.g., threading, snap-fit, etc.) between the insertion tube 108 and the body portion 718 of the hemostasis device 104.

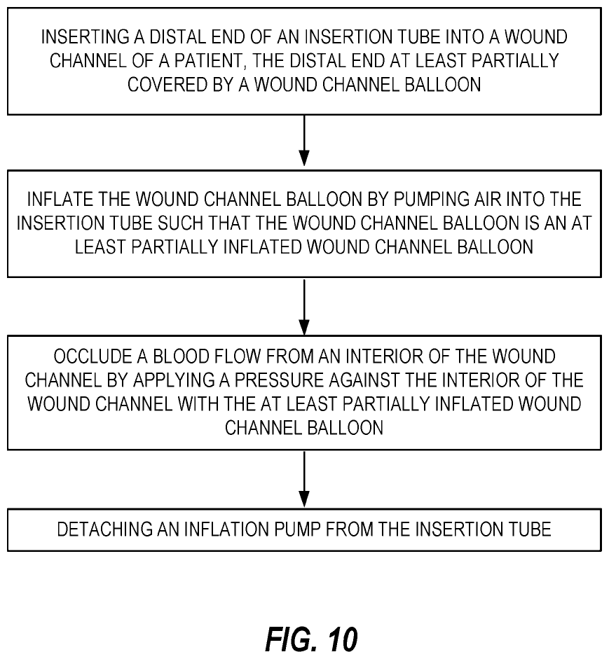

Turning to FIG. 10, an example method 1000 for performing a hemostasis procedure for a wound 110 is depicted. The method 1000 in FIG. 10 can be performed by the systems and devices depicted in FIGS. 1-9 or 11A-15D.

In some examples, at a first operation 1002, the method 1000 includes inserting a distal end of an insertion tube 108 into a wound channel 111 of a patient, the distal end at least partially covered by a wound channel balloon 116. At a second operation 1004, the method 1000 can inflate the wound channel balloon 116 by pumping air into the insertion tube 108 such that the wound channel balloon 116 is an at least partially inflated wound channel balloon 116. At a third operation 1006, the method 1000 can occlude a blood flow from an interior of the wound channel 111 by applying a pressure against the interior of the wound channel 111 with the at least partially inflated wound channel balloon 116. Occluding the blood the blood flow at the third operation 1006 can include one or more of: providing an antibiotic to the interior of the wound channel 111; providing an analgesic to the interior of the wound channel 111; coagulating the blood flow; or sealing the interior of the wound channel 111 with a glue (e.g., a mollusk-based glue). Any of these occlusion operations can be performed with a substance (e.g., an antibiotic, an analgesic, a coagulant, a glue, etc.) carried in a collagen-based matrix (e.g., a bovine collagen) on a surface of the wound channel balloon 116. At a fourth operation 1008, the method 1000 can include detaching an inflation pump 404 from the insertion tube 108.

It is to be understood that the specific order or hierarchy of steps in the method(s) 1000 depicted in FIG. 10 and throughout this disclosure are instances of example approaches and can be rearranged while remaining within the disclosed subject matter. For instance, any of the operations depicted in FIG. 10 and throughout this disclosure may be omitted, repeated, performed in parallel, performed in a different order, and/or combined with any other of the operations depicted in FIG. 10 and throughout this disclosure.

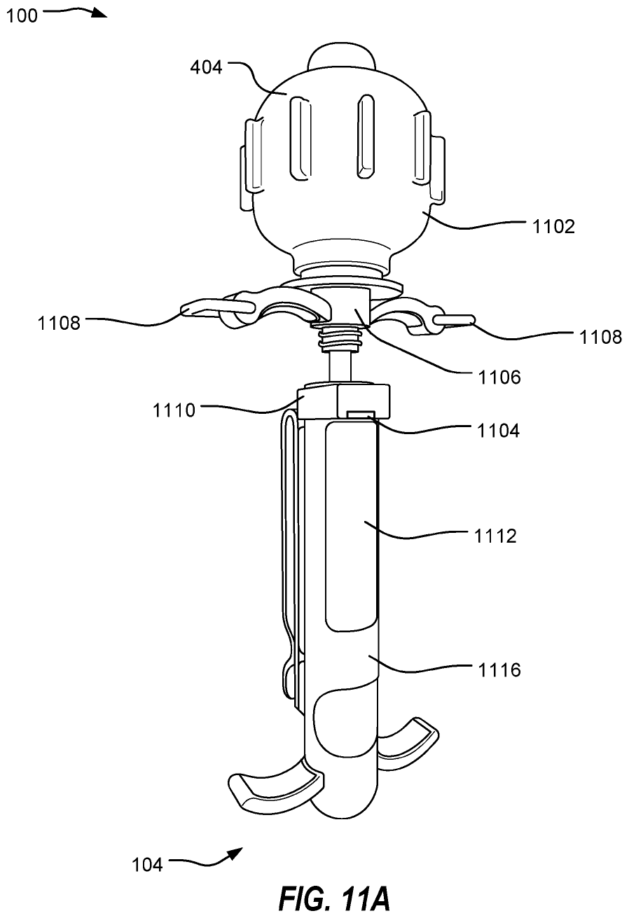

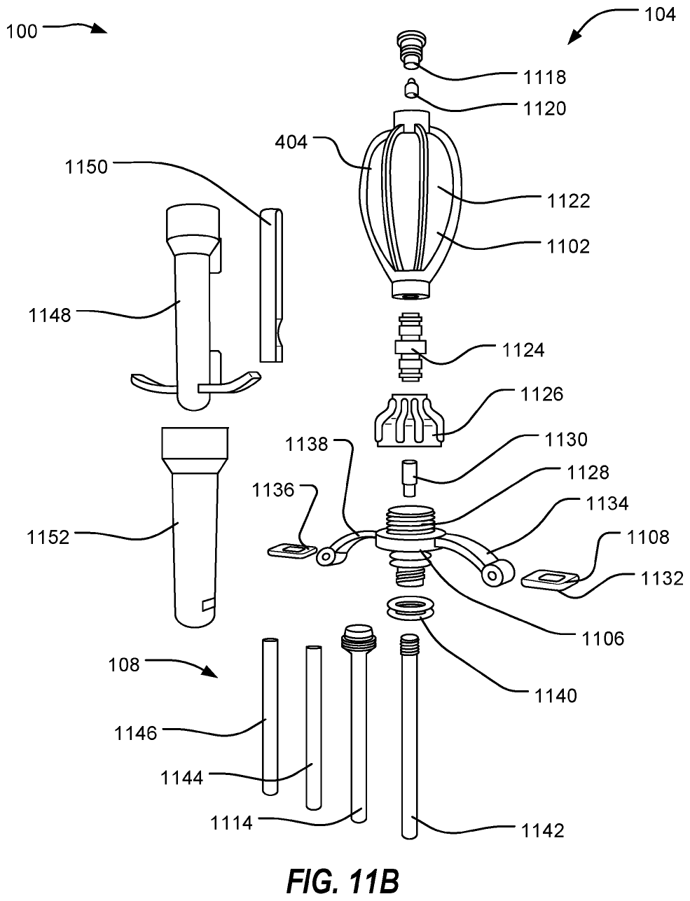

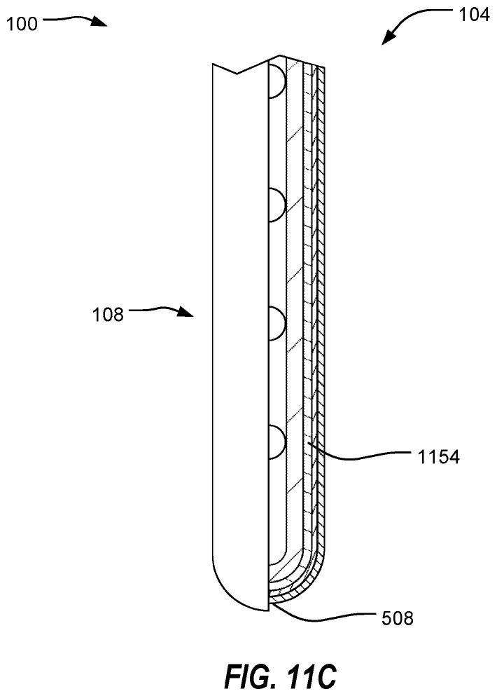

FIGS. 11A-11C illustrate an example hemostasis device 104 and components thereof, which can form at least a portion of the methods and systems disclosed herein regarding FIGS. 1-10 and 12A-15D.

For instance, as depicted in FIG. 11A, the hemostasis device 104 can include a color-coded, removable pump 1102 (e.g., the inflation pump 404) extending from a top distal end of the hemostasis device 104. Furthermore, an electrical impedance sensor 1104 can be disposed in a base portion 1106 (e.g., and/or the insertion tube portion 108) of the hemostasis device 104. The hemostasis device 104 can also include a belt attachment ring 1108, or two belt attachment rings protruding from opposite sides of the base portion 1106. A Bluetooth transceiver 1110, or other type of wireless communication module, can be formed into the base portion 1106 and/or a case 1112 for the insertion tube 108. Moreover, the hemostasis device 104 can have a medicated balloon 1114 (e.g., the wound channel balloon 116) disposed around the insertion tube 108 and insertable into the case 1112 or cover. The medicated balloon 1114 can have a multi-medicative formula for stopping bleeding and/or stabilizing a wound 110 (e.g., a gunshot, stab wound, or shrapnel wound in a field environment), as discussed below regarding FIG. 11C. The case/cover 1112 (e.g., and/or the base portion 1106 of the insertion tube 108) can include a GPS sensor 714 or transmitter, which can be used to locate and extract the patient from the field. Furthermore, the hemostasis device 104 can include a rubber-coated case 1116 for covering and protecting the medicated balloon 1114. A formula label can be disposed (e.g., printed, engraved, etc.) on the rubber-coated case 1116 indicating a type of medication on the medicated balloon 1114 and/or other details of the hemostasis device 104 (e.g., instructions, dimensions, intended injury type for usage, etc.). The hemostasis device 104 can also include one or more grips, such as a “lizard tongue” grip including two curved members extending away from opposite sides of the case, near a distal end of the case (e.g., the grip arm(s) 704). These grip arm(s) 704 can be used to pull the case away from the medicated balloon 1114, which can be snap or friction fitted to the case 1112 at the base, thus exposing the medicated balloon 1114 for use.

FIG. 11B illustrates additional components of the hemostasis device 104 disclosed herein. For instance, the hemostasis device 104 can include a check valve body 1118 coupled to a check valve plunger 1120 set into a top or first opening of a ribbed hand pump 1122 (e.g., the inflation pump 404). Additionally, a second opening opposite the first opening can receive a coupler 1124, which couples the ribbed hand pump 1122 to a threaded connector 1126 which, in turn, couples to a main connector 1128 (e.g., the base portion 1106) of the hemostasis device 104. A one-way valve 1130 can be disposed within the threaded connector 1126, for instance, in a space/cavity defined by a flared portion of the threaded connector 1126 and a receiving area of the main connector 1128 (e.g., protruding from a top surface of the main connector 1128). A first square loop 1132 can form a first belt attachment ring by coupling to a first grip member 1134 extending from a first side of the main connector 1128, and a second square loop 1136 can form a second belt attachment ring by coupling to a second grip member 1138 extending from a second side of the main connector 1128, opposite the first side. One or more O-rings 1140 can couple to a lower connector portion extending from the main connector 1128 to form a seal with the medicated balloon 1114 disposed over a guide rod 1142, which together form the insertion tube 108. An elastic fiber sock 1144 and/or coating can be disposed over the medicated balloon 1114 (e.g., to provide tear-resistance, increased strength, medication carrying ability, and/or wound penetration for the medicated balloon 1114). Additionally, a medicated collagen 1146 can be disposed on the elastic fiber sock 1144. For instance, the elastic fiber sock 1144 can be a carrier for antibiotics, blood clotting agent(s) (e.g., thrombin, lipids or ions that react with the blood), analgesic(s) (e.g., ketamine, lidocaine), stem cells, sealant, and/or lubricant to the wind site. The hemostasis device 104 can further include a container 1148 (e.g., the case/cover 712) which can be placed over and removed from the insertion tube 108, with a belt clip 1150 (e.g., steel) attached to its side. Furthermore, a thermoplastic polyurethane (TPU) 1152 (e.g., plastic cover) having printed information can be placed over the container 1148.

FIG. 11C illustrates additional components of the hemostasis device 104 disclosed herein. For instance, the hemostasis device 104 can include one or more medications 1154 disposed on an exterior of the insertion tube 108 (e.g., the medicated balloon 1114, the elastic fiber 1144, the medicated collagen 1146, etc.). The medication(s) can include any combination of an antibiotic (e.g., Vancomycin), a hemostatic agent (e.g., Surgicel), an anti-inflammatory compound (e.g., Toradol), a nerve pain medication (e.g., Gabapentin), and/or an adhesive/epoxy or other stabilizing agent.

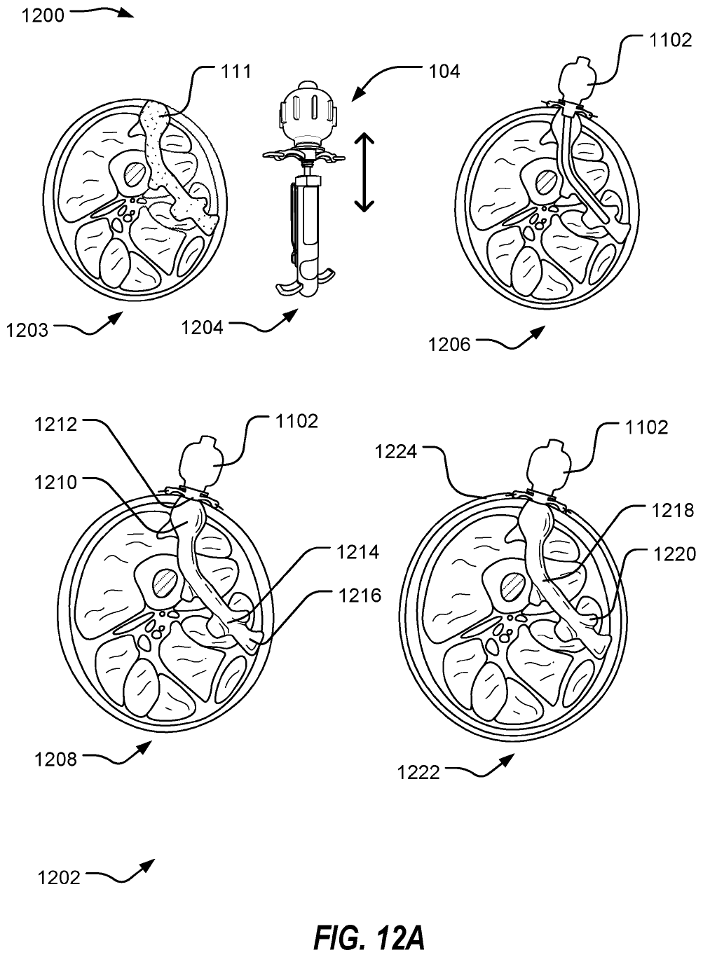

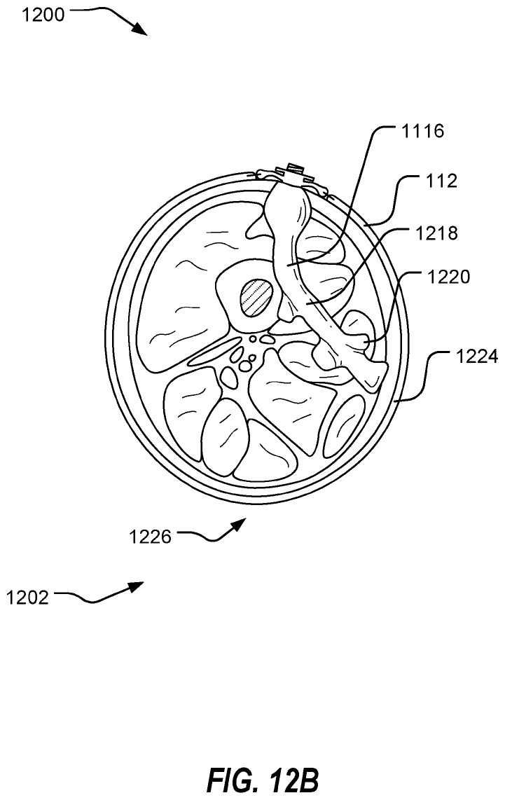

FIGS. 12A and 12B illustrate an example system 1200 for performing a hemostasis procedure 1202 with a hemostasis device 104 for an injured leg 1203, including multiple procedure steps, which can form a part of the methods and systems disclosed herein regarding FIGS. 1-11C or 13-15D.

For instance, the system 1200 and/or method 1202 can include a first step 1204 in which, after a wound 110 occurs (e.g., a gunshot wound 110), the insertion tube 108 (e.g., the “stick”) is removed from a protective sleeve 106, such as the case/cover/container 712/1148. A second step 1206 can include inserting the insertion tube 108 into the wound 110. A third step 1208 can include inflating the wound channel balloon 116 of the insertion tube 108 to apply pressure to interior walls of the wound 110. Inflating the balloon portion can include inflating a first bulb or bulge 1210 at a first end 1212 of the insertion tube 108 near the wound opening, and/or inflating a second bulb or bulge 1214 formed into a distal second end 1216 of the insertion tube 108 opposite the first end 1212. Additionally, inflating the balloon portion 116 can include inflating a primary stem 1218 of the balloon 116 as well as one or more sub-branches 1220 splitting from the primary stem 1218 (e.g., formed by shrapnel, debris, etc.). A fourth step 1222 can include wrapping the belt 1224 around the leg 1204 (e.g., or other wounded body part) of the patient, and tightly securing the belt 1224. A fifth step 1226 (e.g., depicted in FIG. 12B) can include removing the pump 1102 from the hemostasis device 104. Upon performing these steps, the wound channel balloon 116 (e.g., with the tear resistant elastic coating) can go around the bone fragments and/or lodged bullet while slightly pressing against the bone fragments, lodged bullet, and/or interior surfaces of the wound 110.

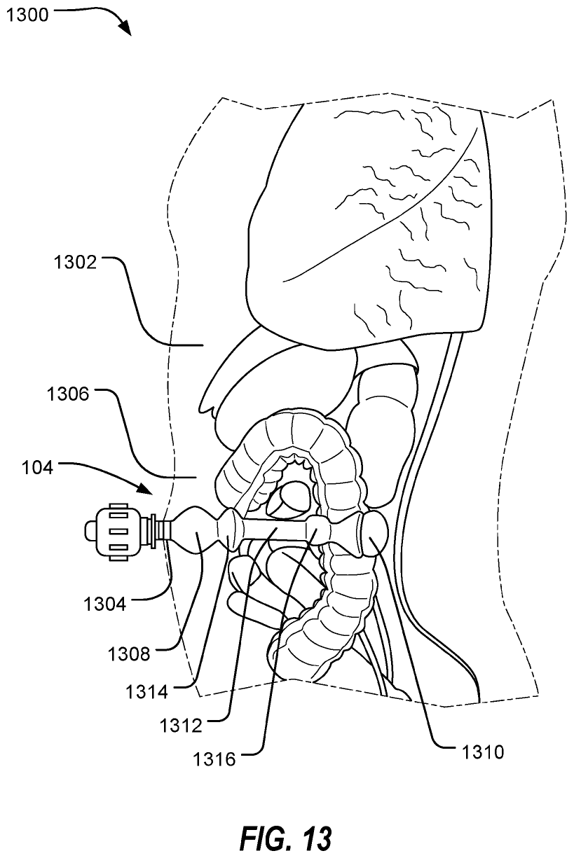

FIG. 13 illustrates an example system 1300 for performing a hemostasis procedure at an injured abdomen 1302, which can form at last a portion of the system(s) disclosed herein regarding FIGS. 1-12B or 14-15D. For instance, the wound site 1304 can be an upper quadrant 1306 (e.g., an upper right quadrant) of the abdomen 1302 of the patient. The hemostasis device 104 may be configured for usage in the abdomen 1302, for instance, with a length dimension extending a longer distance for the deeper wound penetration occurring in the abdomen. Additionally or alternative, the hemostasis device 104 can include one or more bulges or bulb portions, such as the first bulge 1308 adjacent to the pump 1102 at the wound opening, a second bulge 1310 at a distal end of the insertion tube 108 for expanding at a terminal end of the wound 110, and/or one or more intermediary bulges along the length 1312 of the insertion tube 108 (e.g., a third bulge 1314 adjacent to the first bulge 1308 and/or a fourth bulge 1316 adjacent to the second bulge 1310). The one or more bulge(s) 1310, 1312, 1314, and/or 1316 can occlude blood flow while also operating to keep the hemostasis device 104 in place.

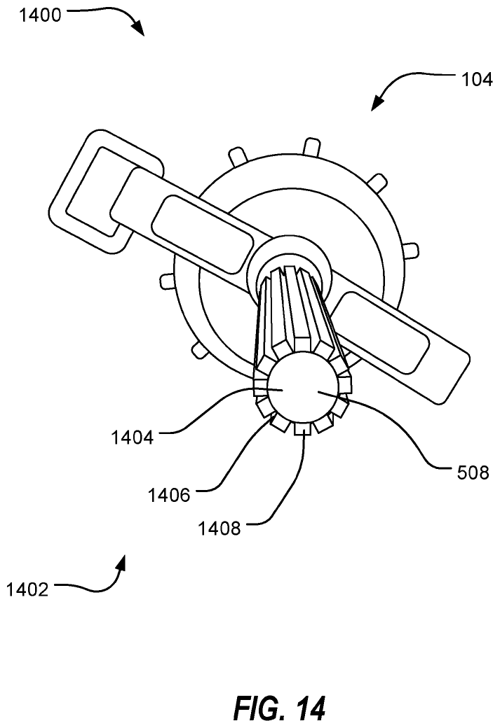

FIG. 14 illustrates an example system 1400 for performing a hemostasis procedure from a bottom perspective view 1402. In some examples, the hemostasis device 104 can include a grooved sock 1404 (e.g., elastic fiber sock 1144) covering the wound channel balloon 116 and/or the insertion tube 108 or guide rod 1142. The grooves 1406 can be substantially evenly distributed radially around the grooved sock 1404, and/or can run a length of the grooved sock 1404. The grooved sock 1404 can be a contoured fiber matrix layer containing/holding a carrier agent such as a collagen gel 1408. The collagen gel 1408 can carry and or deliver antibiotics, blood clotting agent(s) (e.g., thrombin, lipids or ions that react with the blood), analgesic(s) (e.g., ketamine, lidocaine), stem cells, sealant, and/or lubricant directly to the damaged area of the arteries and other soft tissues of the internal wound channel 111. The grooved sock 1404 can also carry lubricant to lubricate the wound 110 such that there is reduced damage caused by removal of the hemostasis device 104 and no lasting residues or clean up needed.

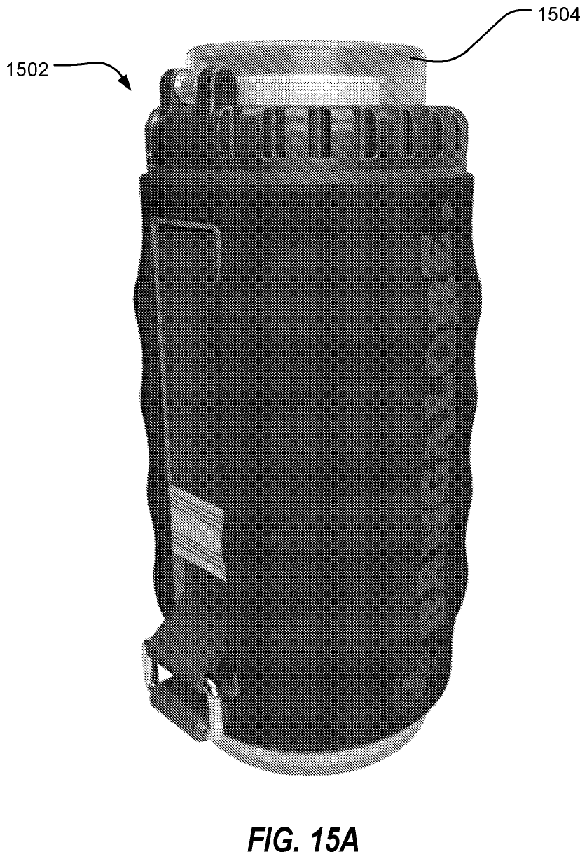

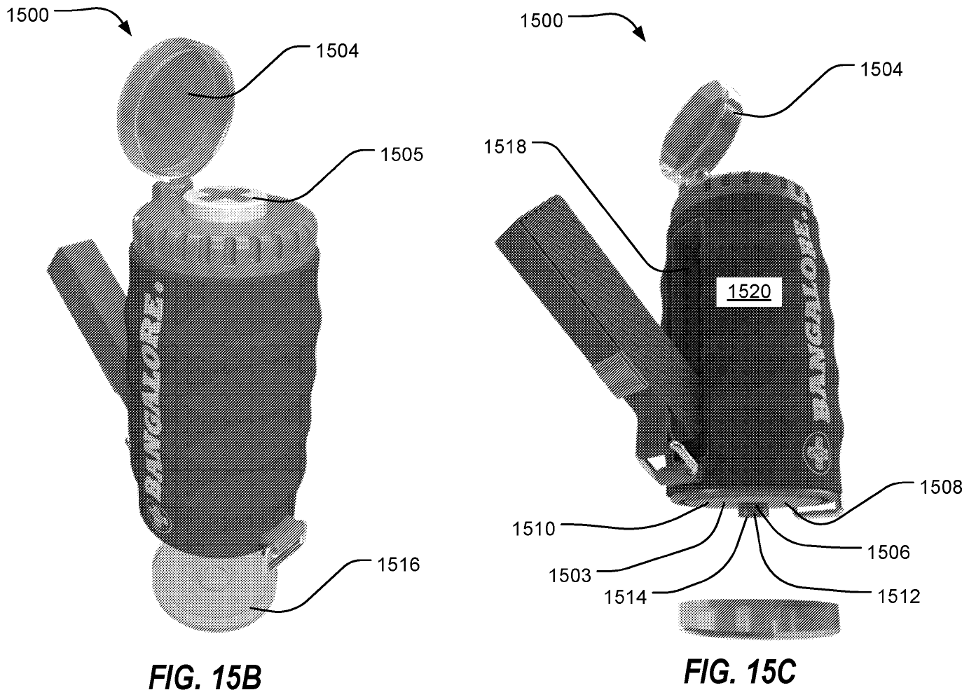

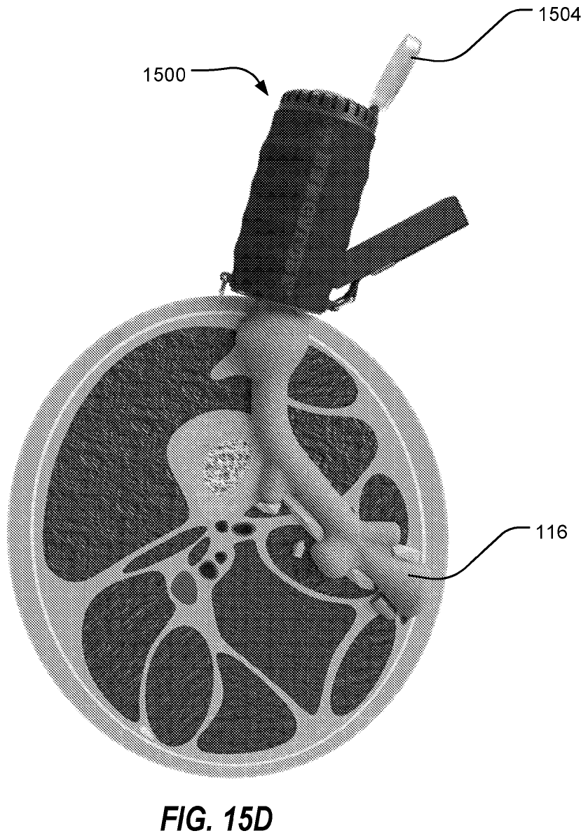

FIGS. 15A-15D illustrate an example hemostasis device 104 and components thereof, which can form at least a portion of the methods and systems disclosed herein regarding FIGS. 1-14.

In some examples, the hemostasis device 104 can include a storage canister 1502 which can house an uninflated wound channel balloon 116 prior to usage. For instance, the hemostasis device 104 can omit the insertion tube 108 portion and/or can house the wound channel balloon 116 in a compressed state when in a storage or carrying configuration. During the hemostasis procedure 1202, the storage canister 1502 can be pressed and/or sealed against and/or over the wound 110. A cover 1504 (e.g., an activator cap, a top lid, a side panel, etc.) can be opened to expose an activation mechanism 1505 (e.g., an activator or a button). Upon receiving a force at the activation mechanism, a compressed gas (e.g., from a carbon dioxide cartridge) can be released into the wound channel balloon 116, causing it to inflate into the wound channel 111. Furthermore, one or more medication beads can be disposed in the storage canister 1502 below and/or adjacent to the wound channel balloon 116, for instance, in a bead storage pocket formed into a bottom portion of the storage canister 1502. The release of the compressed gas and/or the expansion of the wound channel balloon 116 can cause the medication beads to be pushed into the wound channel 111 in front of/prior to the wound channel balloon 116 entering the wound channel 111. The medication beads can be formed from a powder into small pellets or BBs of medication, and can include one or more of antibiotic beads, clotting medication beads, and/or anesthesia beads. In some scenarios, usage of medication beads rather than powders/liquids can avoid clumping and improve delivery of the medications. Furthermore, the wound channel balloon 116 can include a coating of an adhesive or sealant on its outer surface to secure the inflated wound channel balloon 116 into the wound channel 111. The wound channel balloon 116 can be a non-compliant balloon in that it may omit substantial preformed structural components. In this way, the non-compliant wound channel balloon 116 can follow the wound channel 111 as a path of least resistance, regardless of the shape of the wound channel 111 (e.g., an irregular shape). The wound channel balloon 116 can be formed of a flexible material such as silicone. Additionally, the storage canister 1502 can include a rubber sleeve forming an outer surface (e.g., a gripping surface), and can include one or more contours for gripping or printed instructions.

Moreover, in some instances, the hemostasis device 104 can include a sealing ring 1506 formed into a bottom portion 1508 (e.g., a contact surface 1510) of the storage canister 1502. The sealing ring 1502 can include an insertion lip or protrusion 1512 (e.g., a circular ring) extending from the bottom portion 1508 with an extension distance being between 1/10 inches and 5 inches (e.g., ¼ inch). The storage canister 1502 can include an adhesive/sticker 1503 (e.g., a radial adhesive) as well as an adhesive cover (e.g., a thin paper or tape), which can be peeled off of the adhesive/sticker 1503 prior to use to expose the adhesive/sticker 1503. The adhesive/sticker 1503 can be disposed on the contact surface 1510 around the insertion lip or protrusion 1512 and/or an edge of the storage canister insertion lip or protrusion 1512. As such, the adhesive/sticker 1503 can form a seal around the wound opening when the contact surface 1510 is pushed against the wound opening. With the insertion lip or protrusion 1512 in place at the wound opening and the contact surface 1510 pushed against the skin around the wound opening, the adhesive/sticker 1503 can secure the hemostasis device 104 in place so that the hemostasis device 104 can guide the wound channel balloon 116 into the wound channel 111.

Furthermore, the bottom opening 1514 from which the wound channel balloon 116 is expelled can be covered by a lid 1516 which can be removed prior to use. Additionally, the bottom opening 1514 can be at least partially closed or sealed by a membrane, such as a collagen membrane, which can be broken by the pressure of the wound channel balloon 116 and/or the release of the compressed gas, thus clearing the path for the wound channel balloon's 116 expansion into the wound channel 111.

In some scenarios, the storage canister 1502 can include a belt storage compartment 1518 formed into a side 1520 of the storage canister 1502 for housing the holding strap 112. The holding strap 112 can be pre-attached to one of the square loops 1132 or 1136 adjacent to the storage compartment 1518 and/or can be unattached to the other square loop 1132 or 1136. To use, the holding strap 112 can be pulled out of the belt storage compartment 1518 to be wrapped around the injured user and connected to the other square loop 1132 or 1136. Additionally or alternatively, the holding strap 112 can include a hook-and-loop portion to simplify the securing process.

In some instances, the hemostasis device 104 can include a haptic feedback assembly to provide a haptic indication to the user. The haptic feedback assembly can include a vibrator or other type of mechanical actuator to create pulses or vibrations at the wound site and/or on an outer surface of the hemostasis device 104 (e.g., being held by the user's hand). The pulses and/or vibrations can simulate a heartbeat rate (e.g., 98 bpm) or other type of stimulation, which can calm the user and/or assist the user in lowering their own heartbeat. This can be beneficial for users who are undergoing shock and are experiencing a rapid heartbeat which can increase their blood loss.

While the presently disclosed technology has been described with reference to various implementations, it will be understood that these implementations are illustrative and that the scope of the presently disclosed technology is not limited to them. Many variations, modifications, additions, and improvements are possible. More generally, implementations in accordance with the presently disclosed technology have been described in the context of particular implementations. Functionality may be separated or combined differently in various implementations of the disclosure or described with different terminology. These and other variations, modifications, additions, and improvements may fall within the scope of the disclosure as defined in the claims that follow.

1. A hemostasis device including:
an insertion tube extending with a distal end and one or more openings formed into a surface of the insertion tube;
an inflation pump couplable to the distal end of the insertion tube for pumping air into an inner channel of the insertion tube; and
a wound channel balloon disposed at least partially around the insertion tube such that an actuation of the inflation pump causes the air to flow from the inner channel through the one or more openings and inflate the wound channel balloon.

2. The device of claim 1,
further including,
a holding strap coupling to a first side of the insertion tube and a second side of the insertion tube.

3. The device of claim 1,
wherein,
the insertion tube has at least one of a flexible portion or an articulated portion.

4. The device of claim 1,
wherein,
the wound channel balloon includes expansion fingers configured to expand into wound features in response to the inflation pump inflating the wound channel balloon.

5. The device of claim 1,
further including,
a gauge fluidly coupled to the insertion tube to indicate a pressure corresponding to an interior of the wound channel balloon.

6. The device of claim 1,
further including,
one or sensors to generate data related to one or more health parameters; and
a wireless interface for transmitting the data related to one or more health parameters.

7. The device of claim 6,
wherein,
the wireless interface is a Bluetooth interface.

8. The device of claim 1,
further including,
a global positioning system (GPS) system; and
a cover such that removal of the cover from the insertion tube triggers the GPS system to generate and transmit location data.

9. The device of claim 1,
further including,
a collagen gel carrier agent disposed on an outer surface of the wound channel balloon, the carrier agent including at least one of: an antibiotic, an analgesic, a coagulant, or a glue.

10. The device of claim 9,
wherein,
the carrier agent is a bovine-based collagen matrix.

11. The device of claim 9,
wherein,
the carrier agent includes the glue, and the glue is a mollusk-based glue.

12. A hemostasis device including:
a flexible insertion tube including one or more air pathway openings fluidly coupling an inner channel of the flexible insertion tube to an inflation space defined between an outer surface of the flexible insertion tube and a wound channel balloon;
the wound channel balloon disposed at least partially over the flexible insertion tube;
a first coupling mechanism for fluidly coupling an inflation pump to the flexible insertion tube; and
a second coupling mechanism for removably coupling a cover over the flexible insertion tube.

13. The device of claim 12,
further including,
the inflation pump couplable to a first end of the flexible insertion tube such that the inflation pump is fluidly coupled to the inflation space between the outer surface of the flexible insertion tube and the wound channel balloon, actuations of the inflation pump expanding the inflation space.

14. The device of claim 12,
further including,
one or more gripping arms extending from the hemostasis device between the first coupling mechanism and the second coupling mechanism providing one-handed removal of the hemostasis device.

15. The device of claim 12,
further including,
the cover including one or more gripping arms providing one-handed access for pulling the cover away from the wound channel balloon and the flexible insertion tube.

16. A method of performing wound hemostasis comprising:
inserting a distal end of an insertion tube into a wound channel of a patient, the distal end at least partially covered by a wound channel balloon;
inflating the wound channel balloon by pumping air into the insertion tube such that the wound channel balloon is an at least partially inflated wound channel balloon; and
occluding a blood flow from an interior of the wound channel by applying a pressure against the interior of the wound channel with the at least partially inflated wound channel balloon.

17. The method of claim 16,
wherein,
inflating the wound channel balloon includes actuating an inflation pump fluidly coupled to the wound channel balloon;
and the method further includes detaching the inflation pump from the insertion tube.

18. The method of claim 16,
wherein,
an outer surface of the wound channel balloon is coated with a collagen-based matrix, and occluding the blood flow further includes providing an antibiotic and an analgesic to the interior of the wound channel with the collagen-based matrix.

19. The method of claim 18,
wherein,
occluding the blood flow of the interior of the wound channel further includes at least one of:
coagulating the blood flow with a coagulant carried by the collagen-based matrix; or
sealing the interior of the wound channel with a mollusk-based glue.

20. The method of claim 19,
wherein,
occluding the blood flow of the interior of the wound channel includes securing a holding strap around a portion of a person receiving a device including the insertion tube, the holding strap being coupled to a first side and a second side of the device.
